(19) United States
(12) Patent Application Publication
(10) Pub. No.: US 2023/0343448 A1
(43) Pub. Date: Oct. 26, 2023

(54) SYSTEM AND METHOD FOR TRACEABILITY AND HYGIENE COMPLIANCE MANAGEMENT REPROCESSING OF REUSABLE MEDICAL DEVICES AND ENVIRONMENTS

(71) Applicant: Nanosonics Limited, Lane Cove West, NSW (AU)

(72) Inventors: Baheerathan Gnanasundram, Lane Cove West, NSW (AU); Prabhanjan Batni, Lane Cove West, NSW (AU); Jodie Gunns, Lane Cove West, NSW (AU)

(73) Assignee: Nanosonics Limited, Lane Cove West, NSW (AU)

(21) Appl. No.: 18/007,652

(22) PCT Filed: Jun. 3, 2021
(86) PCT No.: PCT/IB2021/000386

§ 371 Date: Dec. 1, 2022

Related U.S. Application Data
(60) Provisional application No. 63/034,616, filed on Jun. 4, 2020.
(60) Provisional application No. 63/068,481, filed on Aug. 21, 2020.
(60) Provisional application No. 63/172,822, filed on Apr. 9, 2021.

(51) Int. Cl. G16H 40/40; G16H 70/20; A61L 2/28

(52) U.S. Cl. CPC G16H 40/40; G16H 70/20; A61L 2/28; A61L 2202/24

(57) ABSTRACT

A method for reusable medical device reprocessing compliance and system for hygiene standards compliance of an environment or a device. For instance, in the method, a reusable medical device is reprocessed before performing a second medical procedure and after performing a first medical procedure using a reprocessing procedure and reprocessing data is uploaded. The method a first medical procedure is performed using a reusable medical device and first medical procedure data is uploaded. The reusable medical device is interactively validated before performing a second medical procedure, including querying for procedure details providing instructions. The second medical procedure is performed using the reusable medical device and second medical procedure data is uploaded. An alert is automatically generated based on comparing the first medical procedure data, the procedure details, the second medical procedure data and the reprocessing data with hygiene standards. The alert indicates noncompliance with hygiene standard(s).

### CROSS-REFERENCE TO RELATED APPLICATION(S)

This application is a national stage application filed pursuant to 35 U.S.C. § 371 of International Application No.: PCT/M2021/000386, which was filed on Jun. 3, 2021 and claims the benefit of U.S. Provisional Patent Application Ser. No. 63/034,616, filed Jun. 4, 2020, U.S. Provisional Patent Application Ser. No. 63/068,481, filed Aug. 21, 2020, and U.S. Provisional Patent Application Ser. No. 63/172,822, filed Apr. 9, 2021, the entirety of each of which are incorporated by reference herein.

### TECHNICAL FIELD

The present disclosure generally relates to reusable medical devices and environments, and more particularly to systems and method for traceability and hygiene compliance management of the reprocessing of reusable medical devices and environments to required laws, standards, guidelines or internal standard operating procedures.

### BACKGROUND

Traditional traceability systems are anchored in the OR/Central Sterilization Department, where decontamination of reusable medical devices is standardized and centralized. However, there are a growing number of reusable medical devices that sit outside of this environment and a growing number of reprocessing systems and methods that also sit outside of OR/Central Sterilization Department for the benefit of workflow efficiency within medical facilities. Additionally, there is a growing need to manage the traceability of reprocessing and patronage of other medical environments including medical treatment rooms, patient environments and bathrooms, medical transport etc. as well as other facilities and environments that may be used to house or treat patients that pose cross contamination risk. Reprocessing of these reusable medical devices and environments is required to be managed in accordance with laws, standards, guidelines or internal standard operating procedures to reduce risk of cross contamination. Frequently, information related to the reprocessing of reusable medical devices and environments is held in separate, complicated and difficult to manage paper based systems that are frequently mismanaged or lost leading these systems to be ineffective in providing effective traceability and compliance to laws, standards, guidelines or internal standard operating procedures to adequately protect patients by providing needed traceability evidence to manage cross contamination risk and outbreak efficiently.

### BRIEF DESCRIPTION

Therefore and according to one aspect, there is provided a method for reusable medical device reprocessing compliance. The method includes the following steps: Providing a plurality of reusable medical devices. Reprocessing the reusable medical device after performing a first medical procedure using a reprocessing procedure and uploading first medical procedure data of the first medical procedure reprocessing data of the first reusable medical device. Interactively validating the reusable medical device for a second medical procedure before performing the second medical procedure. The interactive validating comprises querying for procedure details of the second medical procedure and providing instructions based on the procedure details of the second medical procedure. Performing the second medical procedure using the reusable medical device and uploading second medical procedure data of the second medical procedure. Automatically generating an alert based on comparing the first medical procedure data of the first medical procedure, the procedure details and the second medical procedure data of the second medical procedure and the reprocessing data with a plurality of hygiene standards, the alert indicating noncompliance with at least one of the plurality of hygiene standards.

In another aspect, there is provided a system for hygiene standards compliance of an environment or a device. The system includes a central application and one or more mobile devices. The one or more mobile devices are configured the perform the following steps: Upload reprocessing data to the central application, the reprocessing data relating to a reprocessing procedure of the environment or the device. Upload first procedure data of a first procedure involving the environment or the device to the central application. Interactively validate the environment for a second procedure before performing the second procedure. The interactive validating comprises querying for procedure details of the second procedure and providing instructions based on the procedure details of the second procedure. Upload second procedure data of a second procedure involving the environment or the device to the central application. The central application automatically generates an alert based on comparing the first procedure data of the first procedure, the procedure details and the second procedure data of the second procedure and the reprocessing data with a plurality of hygiene standards. The alert indicates noncompliance with at least one of the plurality of hygiene standards.

In another aspect, there is provided a method for demonstrating reusable medical device reprocessing compliance to a patient. The method includes the following steps: Reprocessing a reusable medical device after performing a first medical procedure using a reprocessing procedure and uploading first reprocessing data of the reusable medical device. Interactively validating the reusable medical device for a second medical procedure before performing the second medical procedure, the interactive validating comprising querying for procedure details of the second medical procedure and providing instructions based on the procedure details of the second medical procedure. Receiving a validation message confirming that the reusable medical device complies with hygiene standards. Displaying the validation message to the patient. Performing the second medical procedure using the reusable medical device and uploading second medical procedure data of the second medical procedure. Automatically generating an alert based on comparing first medical procedure data of the first medical procedure, the procedure details and the second medical procedure data of the second medical procedure and the reprocessing data with a plurality of hygiene standards, the alert indicating noncompliance with at least one of the plurality of hygiene standards.

An advantage obtained by the herein described system and method is that the digitized, automated system processes data related to both the reprocessing of the reusable medical devices and/or environment prior to usage and patient procedure information to provide time sequenced capture and correlation. The foregoing allows the system to compare against governing hygiene laws, standards and guidelines to document compliance to provide traceability and also rapidly make known risks of cross contamination for analysis, assessment and management to minimize patient infection risk that may be caused by reusable medical devices and environments not being reprocessed in accordance with governing hygiene laws, standards or guidelines.

The above embodiments are exemplary only. Other embodiments are within the scope of the disclosed subject matter.

### BRIEF DESCRIPTION OF THE DRAWINGS

So that the manner in which the features of the disclosure can be understood, a detailed description may be had by reference to certain embodiments, some of which are illustrated in the accompanying drawings. It is to be noted, however, that the drawings illustrate only certain embodiments and are therefore not to be considered limiting of its scope, for the scope of the disclosed subject matter encompasses other embodiments as well. The drawings are not necessarily to scale, emphasis generally being placed upon illustrating the features of certain embodiments. In the drawings, like numerals are used to indicate like parts throughout the various views.

FIG. 1 is a block diagram depicting a system for traceability and hygiene compliance management of the reprocessing of reusable medical devices and environments to required laws, standards or guidelines, in accordance with one or more aspects set forth herein;

FIGS. 2A & 2B are flowcharts of a traceability and hygiene compliance management system for the reprocessing of reusable medical devices for reprocessing of reusable medical devices including associated high level disinfection reprocessing device cycle data, in accordance with one or more aspects set forth herein;

FIGS. 3A & 3B are flowcharts of a traceability and hygiene compliance management system for the reprocessing of reusable medical devices, in accordance with one or more aspects set forth herein;

FIGS. 4A & 4B are flowcharts of a traceability and hygiene compliance management system for the reprocessing of mobile reusable medical devices, in accordance with one or more aspects set forth herein;

FIGS. 5A & 5B are flowcharts of a traceability and hygiene compliance management system for the reprocessing of mobile reusable medical devices of different types in different environments, in accordance with one or more aspects set forth herein;

FIGS. 6A-6G are graphical user interfaces of a traceability and hygiene compliance management system for the reprocessing of reusable medical devices, in accordance with one or more aspects set forth herein;

FIGS. 7A-7G are graphical user interfaces of a traceability and hygiene compliance management system for the reprocessing of reusable medical devices, in accordance with one or more aspects set forth herein;

FIGS. 8A-8G are graphical user interfaces of a traceability and hygiene compliance management system for the reprocessing of reusable medical devices, in accordance with one or more aspects set forth herein;

FIGS. 9A-9G are graphical user interfaces of a traceability and hygiene compliance management system for the reprocessing of reusable medical devices, in accordance with one or more aspects set forth herein;

FIGS. 10A-10B are graphical user interfaces of a traceability and hygiene compliance management system for the reprocessing of reusable medical devices, in accordance with one or more aspects set forth herein; and

FIG. 11 is a flowchart of a traceability and hygiene compliance management system for the reprocessing of reusable medical devices, in accordance with one or more aspects set forth herein.

Corresponding reference characters indicate corresponding parts throughout several views. The examples set out herein illustrate several embodiments, but should not be construed as limiting in scope in any manner.

### DETAILED DESCRIPTION

The present disclosure relates to techniques for traceability and hygiene compliance management for the reprocessing of reusable medical devices of different types. Advantageously, the present techniques allow for the linkage and aggregation of reusable medical device or environment patient procedure and reprocessing data so that hospitals and other institutions can improve the management of infection prevention processes, procedures, measures and responses. Dashboards and reports can be presented to help manage the reprocessing of mobile or fixed reusable medical devices.

The techniques presented herein provide, in part, documented evidence of the level/type of reprocessing of reusable medical devices and information related to how one or more reusable medical devices was then used to provide documented evidence of compliance with and correlation to governing hygiene laws, standards or guidelines to support facility audit needs, provide transparency, awareness and embody patient protection to reduce risk of cross contamination.

By way of overview, the present disclosure provides a traceability system that is decentralized and capable of supporting reusable medical devices that are located outside of the OR centralized environment, allowing for workflow efficiency within medical facilities. With the present techniques, reprocessing of these reusable medical devices can be managed in accordance with laws, standards and guidelines for patient safety. The systems and methods presented herein allow for information related to the reprocessing of reusable medical devices and information related to usage of reusable medical devices in patient procedures to be combined together to provide effective traceability and compliance to laws, standards and guidelines to adequately protect patients and provide needed traceability evidence to manage cross contamination risk and outbreak. In addition, the present techniques provide the traceability of reprocessing and usage of environments including medical treatment rooms, medical patient environments and other facilities and environments that may be used to house or treat patients that pose a cross contamination risk.

Although the present disclosure is described with respect to certain embodiments, e.g., patients in a healthcare setting, the techniques set forth are equally applicable to other medical or healthcare environments. The techniques presented herein are directed to management of governing hygiene requirements, whether those requirements are laws, standards, guidelines or internal standard operating procedures that are in force in the environments described, again whether related to healthcare or not.

In addition, although certain examples are given with respect to reusable medical devices, this disclosure contemplates application of the same principles and techniques to include environments such as rooms and other locations, and the given examples can be applied to such environments. Similarly, any examples directed to reprocessing of reusable medical devices are equally applicable to the reprocessing or decontamination in the turnover of environments, e.g., to reduce risk of cross contamination between users.

By way of specific example of a problem solved by the present technique, a probe might be used on an HIV positive patient for an inter-operative liver biopsy, the probe's next job may be a belly scan of a patient who is 6 months pregnant with a healthy pregnancy and no risk conditions. By using the present technique, the probe will be prepared for the exposure risk of the patient it will be used upon, i.e., the healthy pregnant woman on a closed skin belly exam. Advantageously, the present technique makes sure that the probe usage will be compliance with hygiene standards, achieving the goal of reprocessing the ultrasound probe following the peri-operative critical exposure in line with critical reprocessing requirements before using upon the healthy woman with intact skin.

In addition, with respect to environmental reprocessing the following examples are achieved using the present technique. Operating rooms may be turned over between operations in compliance with hygiene standards. Patient rooms may be turned over upon death of patient in compliance with hygiene standards, and the turnover many be elevated depending upon the pathogen profile of past patients. Time-based reprocessing may be achieved in compliance with hygiene standards, e.g., a bathroom in a hospital might have a hygiene requirement to be reprocessed every 15 minutes, a patient room may require a deep clean each 2 days, depending on the type of environment, pathogen risk profile and the exposure risk profile. Other examples are that bedrails should be wiped X times per day, floors should be cleaned X times per day, dental rooms should be turned over between each patient; end of life patient environment turnover, COVID patient CT scan environment turn over, etc. These standard operating procedures may be set forth as applicable hygiene standards and tracked and monitored.

### Generalized Example of a System

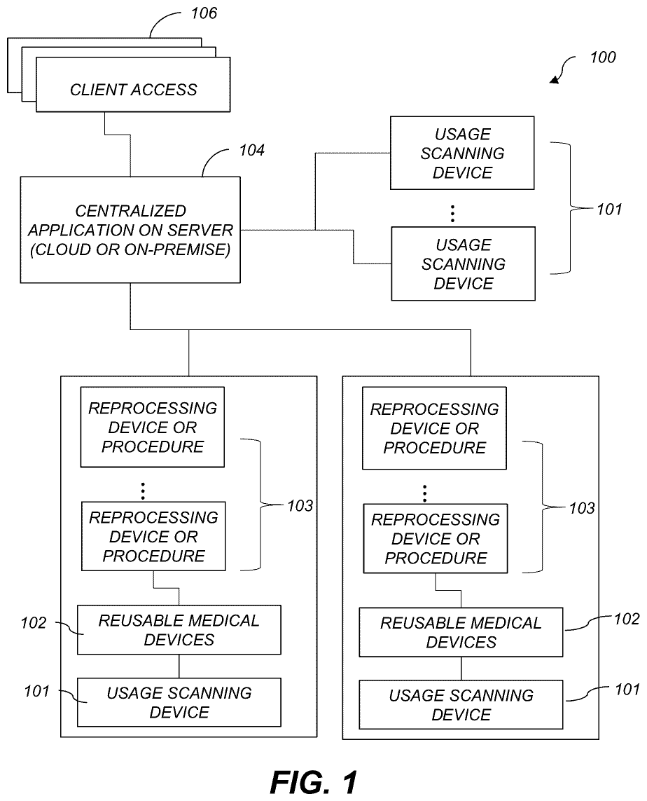

FIG. 1 is a schematic block diagram depicting a system 100 for traceability and hygiene compliance management of reusable medical devices 101, such as ultrasound probes, blood pressure monitors, CT scanners, or other similar devices commonly located in a healthcare facility, such as a hospital, primary caregiver's office, or other care facility. Other non-limiting examples of reusable medical devices include but are not limited to: gamma probes, ophthalmology devices such as tonometers, pachymeters; Doppler ultrasound probes; transoesophageal echocardiography (TOE or TEE) probes; and dental devices. In addition, the present techniques may be used for disinfection of hospital equipment and spaces, dental equipment and spaces, ophthalmology and other medical specialty area in both hospital and clinic environments, as well as aged care facilities, hospices and emergency vehicles and the like.

Other reusable medical devices that may be used in the present technique are listed in the following table:

Bronchoscope (flexible or rigid)
Ultrasound bronchoscope
Bronchoscope accessory
Brush, biopsy, bronchoscope (non-rigid)
Claw, foreign body, bronchoscope (non-rigid)
Curette, biopsy, bronchoscope (rigid 1
Curette, biopsy, bronchoscope (non-rigid)
Forceps, biopsy, bronchoscope (non-rigid)
Forceps, biopsy, bronchoscope (rigid)
Telescope, laryngeal-bronchial
Tube, aspirating, bronchoscope (rigid)
Tubing, Instrumentation, bronchoscope
(brush sheath NO aspirating)
Esophagoscope (flexible or rigid)
Esophagoscope. general & plastic surgery
Esophagoscope, rigid, gastro-urology
Nasopharyngoscope (flexible or rigid)
Laryngoscope, nasopharyngoscope
Mediastinoscope, surgical, and accessories
Duodenoscope and accessories, flexible/rigid
Panendoscope (gastroduodenoscope)
Mini endoscope, gastroenterology-urology
Accessories, cleaning, for endoscopes
Accessories, germicide, cleaning, for endoscopes
High level disinfection reprocessing instrument
for ultrasonic transducers, mist
Washer, cleaner, automated, endoscope
High level disinfection reprocessing instrument
for ultrasonic transducers, liquid
Colonoscope and accessories, flexible/rigid
Choledochoscope and accessories, flexible/rigid
Enteroscope and accessories
Gastroscope and accessories, flexible/rigid
Cystoscope and accessories, flexible/rigid
Ureteroscope and accessories, flexible/rigid
Endoscopic ultrasound system, gastroenterology-urology
Endoscope, neurological
Controller, Temperature, Cardiopulmonary Bypass
System, Thermal Regulating
System, Surgical, Computer Controlled Instrument
Arthroscope

FIG. 1 provides a generalized example of a system 100, but a person of ordinary skill in the art would readily understand that the concepts set forth herein apply to any number of different arrangements of reprocessing reusable medical devices or environments. In particular, in some examples that are within the scope of the present disclosure, multiple reprocessing devices may be located in a fixed reprocessing room or area. In other examples, the reprocessing devices may be portable, and capable of being moved from room to room. Data input related to reprocessing may include automated reprocessing device cycle data directly integrated into the system or manual input of reprocessing effort to document other systems or means to reprocess reusable medical devices or environments. In another aspect, multiple scanning devices may be used by staff or clinicians to track the usage of the reusable medical devices and/or environments in or for patient procedures, with data being uploaded immediately or in a batch mode at a later time after numerous procedures were undertaken.

In the embodiment of FIG. 1, the system 100 includes at least one usage scanning device 101 that is used to track details of usage of one or more reusable medical devices 102, and runs a mobile application. The system 100 includes one or more reprocessing devices 103, that are used to reprocess the reusable medical devices 102. In other examples, the techniques may be embodied as systems or manual methods. For example, a reprocessing device 103 may be an automated ultrasound probe disinfection device, such as a Trophon EPR or Trophon2 device, available from Nanosonics Limited, of Australia. The reprocessing devices 103 may be connected via the hospital network. In such a case, the reusable medical devices 102 may be ultrasound probes that have an RFID or other identification tag, and the reprocessing data referred to in this specification, along with patient procedure information, procedure hygiene classification information, etc., will be uploaded from the reprocessing device 103 directly to the system application. In other examples, the reusable medical device 102 may be one or more endoscopes, Mill machines, CT machines, dental devices, ophthalmology devices, or the like.

In some examples, scanning device 101 may be a tablet, smart phone, smart watch, special-purpose handheld input device, or any other type of device usable to san and track details of usage of one or more reusable medical devices. In other examples, different types of reusable medical devices can be reprocessed with numerous different types reprocessing systems, methods, devices, techniques or equipment, and information on the reprocessing can be loaded either directly to the application by the reprocessing device or by the usage scanning device.

With further reference to FIG. 1, the herein described system 100 includes a server-based application 104, that receives data from the scanning devices 101 and reprocessing devices 103, and allows any number of users 106 to manage the traceability and hygiene compliance of the reusable medical devices 102. The reusable medical devices 102 could be tagged with an RFID tag or may include a barcode, unique identifying alphanumeric code or other such identification information that can be scanned by or manually typed into scanning devices 101. The centralized server application 104 can also offer an applications programming interface (API), described in further detail below. In other embodiments (not pictured), instead of being an ultrasound probe or similar “dumb” device, the reusable medical devices 102 may themselves be smart devices, each equipped a processor and WiFi capability for directly uploading information to the scanning device 101 or the application 104.

In one example, the scanning device 101 may be based on a commercial off-the shelf Android or other smart phone with a pre-loaded mobile application, a dedicated QR-code scanner, and radiofrequency identification (RFID) scanner to scan a tag or code 108. In such a case, the mobile application 104 will provide a user interface (not shown) that allows healthcare personnel to capture details of the reusable medical device, procedure, patient and accession number at the point of care, etc. The scanning device 102 may include USB charging cables and other accessories.

The mobile application running on the scanning device 101 can capture a variety of data about the one or more reusable medical devices 102. For example, the scanning device 101 can capture various data, including but not limited to the following: Medical instrument data via RFID tag; Medical instrument sterilization status; Procedure classification via manual selection using standards and guidelines such as Spaulding classification; and Procedure accession number, patient identifier. A passive tag, such as RFID, can carry a small amount of information which can be used to store such data.

As explained in further detail below, the present techniques are applicable to mobile or moveable reusable medical device and environment reprocessing compliance workflow management by providing end to end traceability between reprocessing device/environment, reprocessing and patient procedure, across all areas of hospitals, clinics and other medical environment for the purpose of reducing the risk of patient cross contamination, thus reducing hospital acquired infections and safeguarding facilities against financial and reputational risk of non-compliance to hygiene standards. Built in to the mobile device is continuous reprocessing compliance training as the application will prompt and warn users as they complete the workflow.

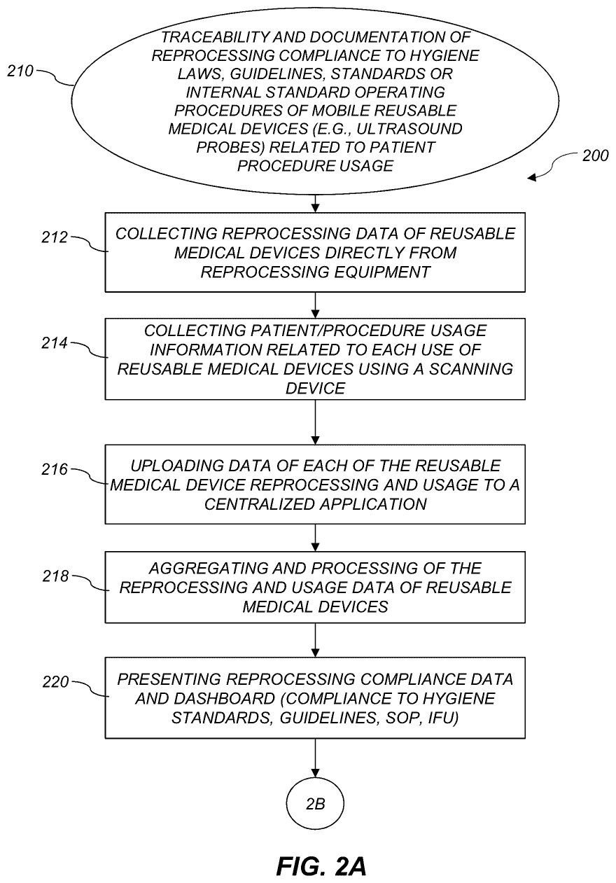

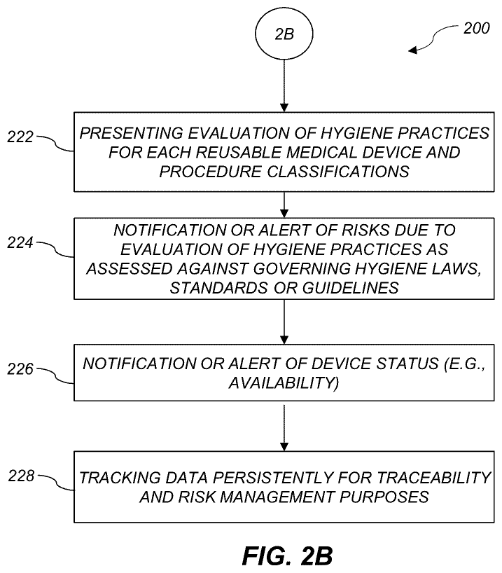

FIGS. 2A-5B provide several working examples or scenarios of the present disclosure.

### Scenario A: High Level Disinfection and Reprocessing Cycle Data

### General Overview

In FIGS. 2A & 2B, an exemplary method 200 utilizes at least one high level disinfection device and associated reprocessing cycle data, a mobile device used by clinicians to collect procedure (i.e., reusable medical device usage data) and a centralized application that combine the reprocessing and usage data to generate a series of compliance dashboards that allow the facility to demonstrate compliance to hygiene standards, guidelines, standard operating procedures (SOPs) and instructions for use (IFUs).

For example, method 200 uses a mobile electronic device that is able to connect to a network using direct connection, Wi-Fi or telecommunications (e.g., 4G, etc.) to send reusable medical device unique identification information, reusable medical device reprocessing information from a high level disinfection reprocessing device, reusable medical device usage information, patient and/or procedure identification information and time and date information to a centralised application that aggregates, processes and presents back evidence based track and traceability information related to the hygiene practices of reusable medical devices, including notifications of hygiene activities that present risk to patient/s as indicated by governing laws, standards, guidelines and product Instructions for Use (IFUs) including:
Unique reusable medical device identification comprising RFID, barcode or other numbering system.
reusable medical device reprocessing data that includes the operator/s who performed the reprocessing, reprocessing device serial number, evidence of reprocessing agent and/or reprocessing device evidence data and time and date of reprocessing processes.
Reusable medical device usage data, which will include information related to the hygiene category of procedure, information related to the clinician or operator of the reusable medical device during usage and date and time of usage.
Patient and/or procedure identification data, which will include information that allows the reusable medical device reprocessing and usages data set be linked back to patient medical or personal record systems for hygiene traceability purposes.

### Scenario A Flowcharts (FIGS. 2A & 2B):

FIGS. 2A & 2B depict an exemplary method 200 for traceability and hygiene compliance management of reprocessing. In one embodiment, at block 210, the method performs the step of traceability and documentation of reprocessing compliance to hygiene laws, guidelines, standards or internal standard operating procedures (SOP) of mobile reusable medical devices (e.g., ultrasound probes) related to patient procedure usage.

For example, at block 212, the method performs the step of collecting reprocessing data of mobile reusable medical devices directly from reprocessing equipment. The capture of the medical procedure data allows for the data to be processed and curated so that the appropriate information is stored in the database (e.g., of the server application) for later determination of infection insights. When each of the medical instruments are reprocessed, the reprocessing information is collected. During reprocessing, the scanning device can continuously reeducate the clinical user on the required hygiene standards, for example by specifying a high-level disinfection (HLD) or a low-level disinfection (LLD) and notifying users if a device has not been reprocessed as it is required to be for use prior to a specific patient procedure. In one specific example, if a user attempts to bypass the proper disinfection procedure by declining to perform an HLD and resets the device to think that a procedure requiring LLD is being done, the system can log this information. As an advantage, the method 200 allows notification of supervisors, for example, if documentation during the procedure falls outside of guidelines, and allowing for rapid course correction or risk management. The reprocessing information may be uploaded to the application. In one example, two distinct data sets may be uploaded to the application, i) the medical procedure data, and ii) the device reprocessing data. In another example, clinician input indicates the level of disinfection or sterilization of the reusable medical device or environment. This data may be uploaded in real-time, or may be uploaded in a batch procedure. If uploaded in a batch procedure, the present system is able to mine the data to find errors in the reprocessing that could lead to alerts that a specific patient may have been exposed to an improperly reprocessed device. In such an embodiment, uploading the reprocessing information includes batch uploading, and correlating of the information comprises sequencing historical procedures and reprocessing events to find the failed or inadequate reprocessing cycle.

Next, the method 200 at block 214 performs the step of collecting patient/procedure usage information related to each use of reusable medical devices using a scanning device. Once collected, the method 200 at block 216 performs the step of uploading data of each of the reusable medical device reprocessing and usage to a centralized application. After the data has been uploaded, the method 200 at block 218 performs the step of aggregating and processing of the reprocessing and usage data of the reusable medical devices. For example, the medical procedure information and the reprocessing information may be correlated to determine if there was a failed or inadequate reprocessing cycle of a specific reusable medical device, and such data can be used to trace potential infections. In other examples, the system supports reporting of key performance indicators (KPIs), and specific corrective actions may be taken depending on the range of acceptable indicators. For instance, the failed or inadequate reprocessing cycle may include a reprocessing cycle insufficient to reprocess the specific medical instrument in view of the specific medical procedure information. In one specific example, correlating the information includes contact tracing of multiple patients exposed to the specific medical instrument with the failed or inadequate reprocessing cycle. In yet another example, correlating the information includes mapping specific levels of disinfection of the medical instruments with each of the medical procedures.

In addition, the system and related method can be used in many ways that are within the spirit of this disclosure. For example, patient information may be uploaded at any time, and the correlations can be re-run to account for the new data. As another example, the correct reprocessing procedure may be dependent not just on the medical procedure previously performed, but also on the condition of the patient. If it is later determined that a patient tests positive for, e.g., COVID-19, the system can retrospectively re-run the correlations to find devices that were not properly reprocessed in light of these new facts. Batch uploaded data, discussed above, can be correlated by determining the time sequence of events and making sure that all protocols have been followed, find any missing disinfection cycles, etc. In such a case, more generally, the method can include uploading updated patient information indicating that at least one patient found in patient information of the medical procedure information has contracted a condition requiring a different level of reprocessing of the medical instruments, and updating the medical procedure information based on the updated patient information.

Subsequent to the aggregation, the method 200 at block 220 performs the step of presenting reprocessing compliance data and dashboard (compliance to hygiene standards, guidelines, standard operating procedures and instructions for use). Also, the method 200 at block 222 performs the step of presenting evaluation of hygiene practices for each reusable medical device and procedure classification. In addition to reporting and dashboards, the method 200 at block 224 performs the step of notification or alert of risks due to evaluation of hygiene practices as assessed against governing hygiene laws, standards or guidelines. In addition, the method 200 at block 226 performs the step of notification or alert of device status (e.g., availability). Then, the method 200 at block 228 performs the step of tracking data persistently for traceability and risk management purposes.

Again, different actions may be taken based on the data collected by the system and employing the described method. For example, patients may be contact-traced and contacted for further testing. In another example, reusable medical devices may be withdrawn from service and scheduled for immediate reprocessing following the correct protocol. In one specific example, if a high-level disinfection was required before a reusable medical device was to be used again, and only a low-level disinfection was performed, the system can trace which reusable medical devices may not have been properly disinfected.

### Scenario B: Mobile Reusable Medical Device Traceability System

### General Overview

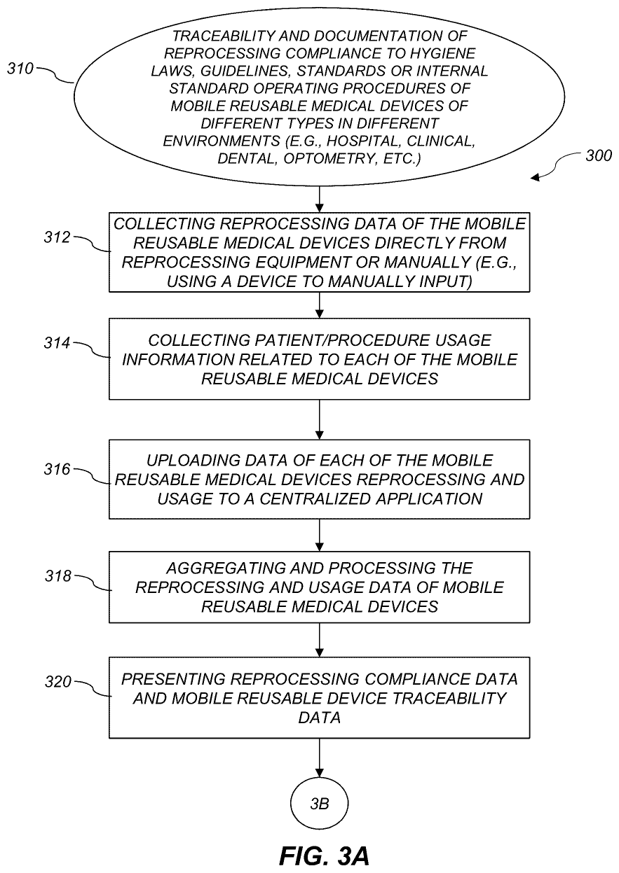

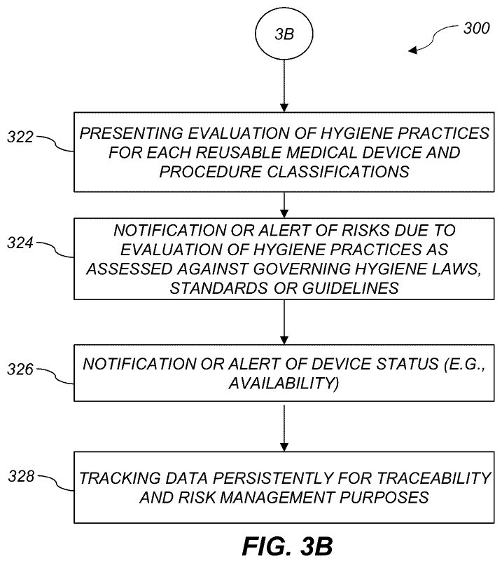

In FIGS. 3A & 3B, an exemplary method 300 is provides a mobile reusable medical device traceability system de-coupled from specific disinfection devices, which is able to pull in data of many reprocessing devices and manual reprocessing systems/processes (e.g., usage in hospitals, clinics, dental practices, optometry practices and other healthcare environments).

For example, method 300 uses a mobile electronic device that is able to connect to a network using direct connection, Wi-Fi or telecommunications (e.g., 4G, etc.) to send reusable medical device unique identification information, reusable medical device reprocessing information, reusable medical device usage information, patient and/or procedure identification information and time and date information to a centralised application that aggregates, process and presents back evidence based track and traceability information related to the hygiene practices of reusable medical devices, including notifications of hygiene activities that present risk to patient/s as indicated by governing laws, standards, guidelines and product IFUs, including:
Unique reusable medical device identification comprising RFID, barcode or other numbering system.
reusable medical device reprocessing data, which includes the operator/s who performed the reprocessing, evidence of reprocessing agent and/or reprocessing device evidence data and time and date of reprocessing processes.
Reusable medical device usage data, which will include information related to the hygiene category of procedure, information related to the clinician or operator of the reusable medical device during usage and date and time of usage.
Patient and/or procedure identification data, which will include information that allows the reusable medical device reprocessing and usages data set be linked back to patient medical or personal record systems for hygiene traceability purposes.

### Scenario B Flowchart (FIGS. 3A & 3B):

The flowcharts of FIGS. 3A & 3B depict the Scenario B example.

For instance, the method 300 at block 310 provides the step of traceability and documentation of reprocessing compliance to hygiene laws, guidelines, standards or internal standard operating procedures of mobile reusable medical devices of different types in different environments (e.g., hospital, clinical, dental, optometry).

In one embodiment, the method 300 at block 312 performs the step of collecting reprocessing data of the mobile reusable medical devices directly from reprocessing equipment or manually (e.g., using a device to manually input). Next, the method 300 at block 314 performs the step of collecting patient/procedure usage information related to each of the mobile reusable medical devices. After collecting the data, the method 300 at block 316 performs the step of uploading data of each of the mobile reusable medical devices reprocessing and usage to a centralized application. After uploading the data, the method 300 at block 318 performs the step of aggregating and processing the reprocessing and usage data of mobile reusable medical devices. Then, the method 300 at block 320 performs the step of presenting reprocessing compliance data and mobile reusable device traceability data. The method 300 at block 322 also performs the step of presenting evaluation of hygiene practices for each reusable medical device and procedure classifications. Then, the method 300 at block 324 performs the step of notification or alert of risks due to evaluation of hygiene practices as assessed against governing hygiene laws, standards or guidelines. Further, the method 300 at block 326 performs the step of notification or alert of device status (e.g., availability). In addition, the method 300 at block 328 performs the step of tracking data persistently for traceability and risk management purposes.

### Scenario C: Fixed Reusable Medical Devices

### General Overview

In the following example, set forth is the additional feature of fixed reusable medical devices and room turnover for these reusable medical devices (including, but not limited to Mill machines, CT machines and the rooms and environments in which they are fixed)

For example, method 400 uses a mobile electronic device that is able to connect to a network using direct connection, Wi-Fi or telecommunications (e.g., 4G, etc.) to send reusable medical device unique identification, unique room or location information, reusable medical device and room or location reprocessing information, reusable medical device and/or room usage information, patient and/or procedure identification information and time and date information to a centralized application that aggregates, processes and presents back evidence based track and traceability information related to the hygiene practices of reusable medical devices and associated rooms or locations and notifications of hygiene activity that present risk to patient as indicated by governing laws, standards, guidelines and product IFUs and notifications of device and/or room availability once reprocessing has been completed including:
Unique reusable medical device identification comprising RFID, barcode or other numbering system.
Unique room or location identification comprising RFID, barcode or numbering system.
reusable medical device and room or location reprocessing data, which includes the operator/s who performed the reprocessing, evidence of reprocessing agent/s, processes and/or reprocessing device evidence data and time and date of reprocessing processes.
reusable medical device usage data, which will include information related to the hygiene category of procedure, information related to the clinician or operator of the reusable medical device during usage and date and time of usage.
Patient and/or procedure identification data, which will include information that allows the reusable medical device reprocessing and usages data set be linked back to patient medical or personal record systems for hygiene traceability purposes.

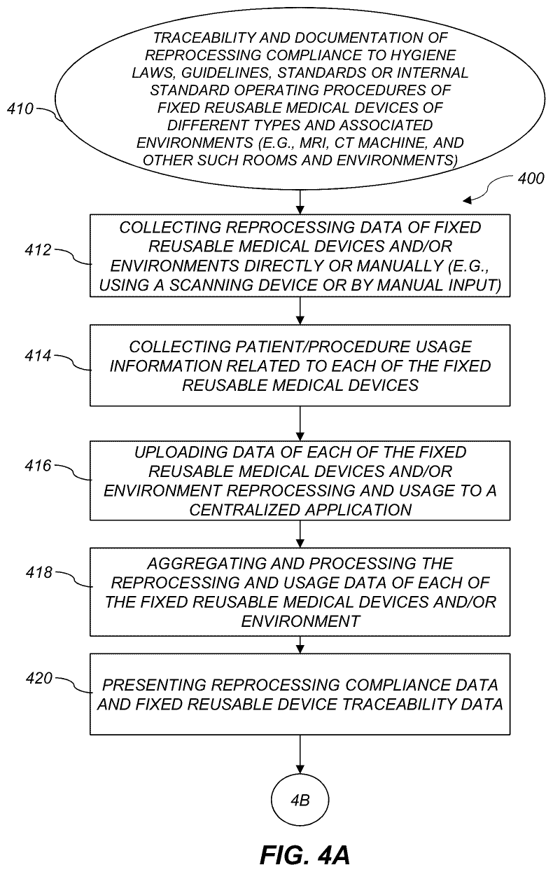

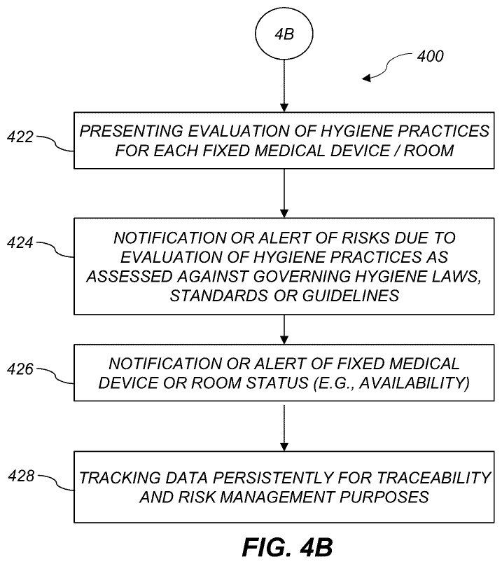

### Scenario C Flowchart (FIGS. 4A&4B):

The flowcharts of FIGS. 4A & 4B depict the Scenario C example.

For instance, the method 400 at block 410 provides the step of traceability and documentation of reprocessing compliance to hygiene laws, guidelines, standards or internal standard operating procedures of fixed reusable medical devices of different types and associated environments (e.g., Mill, CT machine, and other such rooms and environments).

In one embodiment, the method 400 at block 412 performs the step of collecting reprocessing data of the fixed reusable medical devices and/or environments directly or manually (e.g., using a scanning device or by manual input). In addition, the method 400 at block 414 performs the step of collecting patient/procedure usage information related to each of the fixed reusable medical devices. After collecting, the method 400 at block 416 performs the step of uploading data of each of the fixed reusable medical devices and/or environment reprocessing and usage to a centralized application. Next, the method 400 at block 418 performs the step of aggregating and processing the reprocessing and usage data of each of the fixed reusable medical devices and/or environment. Then, the method 400 at block 420 performs the step of presenting reprocessing compliance data and fixed reusable device traceability data. Next, the method 400 at block 422 performs the step of presenting evaluation of hygiene practices for each fixed reusable medical device/room. In addition, the method 400 at block 424 performs the step of notification or alert of risks due to evaluation of hygiene practices as assessed against governing hygiene laws, standards or guidelines. Then, the method 400 at block 426 performs the step of notification or alert of fixed reusable medical device or room status (e.g., availability). Further, the method 400 at block 428 performs the step of tracking data persistently for traceability and risk management purposes.

### Scenario D: Room Hygiene Reprocessing

### General Overview

In the following example, set forth is the additional feature of expanding indication to room hygiene reprocessing and turnover (to include, for example, hospital patient rooms).

For example, the method 500 uses a mobile electronic device that is able to connect to a network using direct connection, Wi-Fi or telecommunications (e.g., 4G, etc. to send unique room or location information, room or location reprocessing information, patient or user information and time and date information to a centralised application that aggregates, process and presents back evidence based track and traceability information related to the hygiene practices of rooms or locations and notifications of hygiene activity that present risk to patients or users as indicated by governing laws, standards, guidelines or infection control requirements and/or room availability once reprocessing has been completed, including:
Unique room or location identification comprising RFID, barcode or other numbering system.
Room or location reprocessing data, which includes the operator/s or cleaners who performed the reprocessing, evidence of reprocessing agent and/or reprocessing device evidence data and time and date of reprocessing processes.
Room or location usage data, which will include information related to date and time of usage.
Patient or user identification data, which will include information that allows usages data set be linked back to patient medical or personal record systems for hygiene traceability purposes. If a patient or user's infection status is known, the patient or user data will include either a specification of or a risk rating indication of the patient's known.

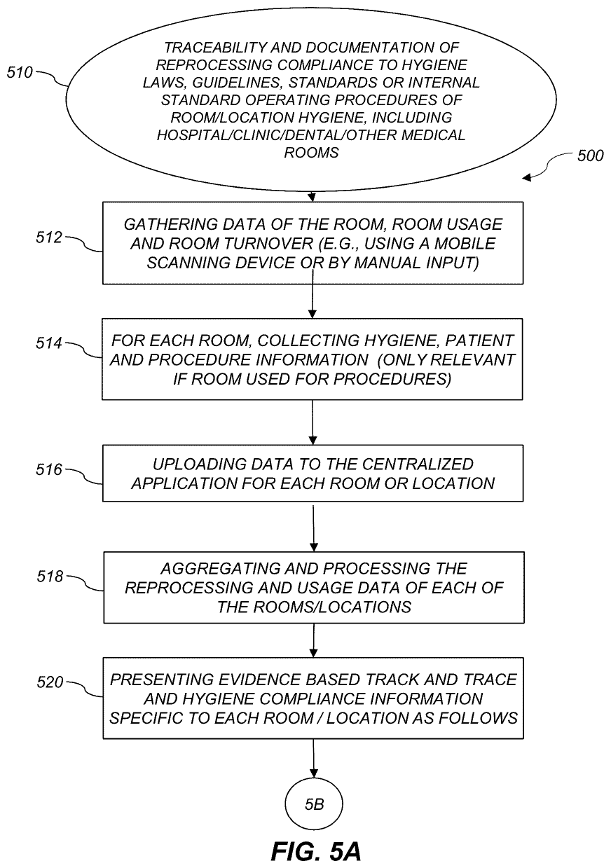

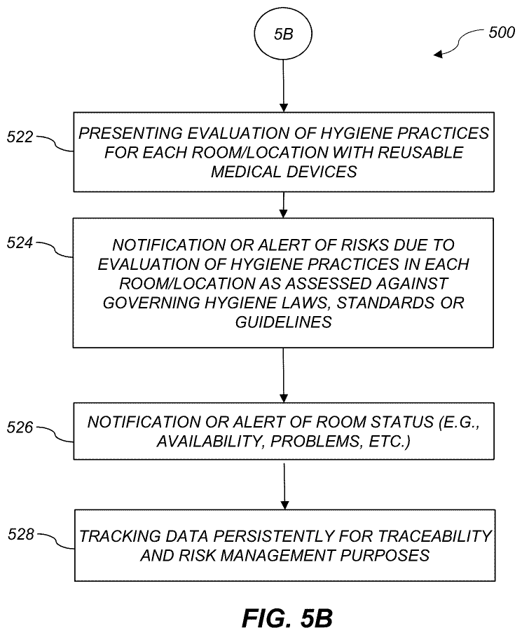

### Scenario D Flowchart (FIGS. 5A & 5B):

The flowcharts of FIGS. 5A & 5B depict the Scenario D example.

For instance, the method 500 at block 510 provides the step of traceability and documentation of reprocessing compliance to hygiene laws, guidelines, standards or internal standard operating procedures of room/location hygiene, including hospital/clinic/dental/other medical rooms, etc.

In one embodiment, the method 500 at block 512 performs the step of gathering data of the room, room usage, and room turnover (e.g., using a mobile scanning device or by manual input). In addition, the method 500 at block 514 performs for each room, the step of collecting hygiene, user, patient and procedure information (only relevant if room used for procedures). After collecting, the method 500 at block 516 performs the step of uploading data to the centralized application for each room or location. Next, the method 500 at block 518 performs the step of aggregating and processing the reprocessing and usage data of each of the rooms/locations. Then, the method 500 at block 520 performs the step of presenting evidence based track and trace and hygiene compliance information specific to each room/location. Subsequently, the method 500 at block 522 performs the step of presenting evaluation of hygiene practices for each room/location with reusable medical devices. Then, the method 500 at block 524 performs the step of notification or alert of risks due to evaluation of hygiene practices in each room/location as assessed against governing hygiene laws, standards or guidelines. In addition, the method 500 at block 526 performs the step of notification or alert of room status (e.g., availability, problems, etc.). Further, the method 500 at block 528 performs the step of tracking data persistently for traceability and risk management purposes.

With respect to all of the examples above, although the system and methods have been described in terms of reusable medical devices and reprocessing, the same concept is applicable to the tracing of required reagents or chemicals that the medical instruments need. For example, if it is later determined that a certain chemical used for reprocessing was contaminated or expired, then this further information can be fed into the system, and the correlations re-run to find any impacted reusable medical devices and patient records, and generate corrective actions and alerts as appropriate.

Another example relates to changing standards of care. For example, if a determination is made that the plan of record is inadequate to prevent infection, information can be updated of new requirements of disinfection for procedures, and all non-compliant reusable medical devices can be identified and scheduled for reprocessing under the changed standard of care.

The techniques described above are appropriate for use in hospitals, clinics, dental offices, as well as facilities that conduct ultrasound procedures, CAT scans, and other procedures. Although the term “reusable medical device” is used throughout, that term can more broadly be used to represent a room having instruments and equipment, with each element of that room requiring its own standards for reprocessing. Different rooms may also be designated with different standards of care depending on if they are within a contaminated area or a clean area, with respect to specific issues such as coronavirus or other infectious diseases.

In addition to correlations finding corrective actions and generating alerts, reporting features are proposed that allow for monitoring KPIs, establishing baselines, determining performance of sites such as hospitals, confirming best practices, etc. For example, data uploaded from multiple hospitals can be correlated to determine the number of mistaken disinfections, for example, and provide a score for that hospital or facility on an automated, weekly basis.

Other devices are ready for use in this system, including stethoscopes, blood pressure sensors, temperature devices, etc., which can be tagged and tracked using the system.

In addition, any of the techniques described herein may be applied to the disinfection of emergency vehicles, including but not limited to ambulances. As one example, the use of validated cleaning agents and other materials may be monitored in emergency vehicles.

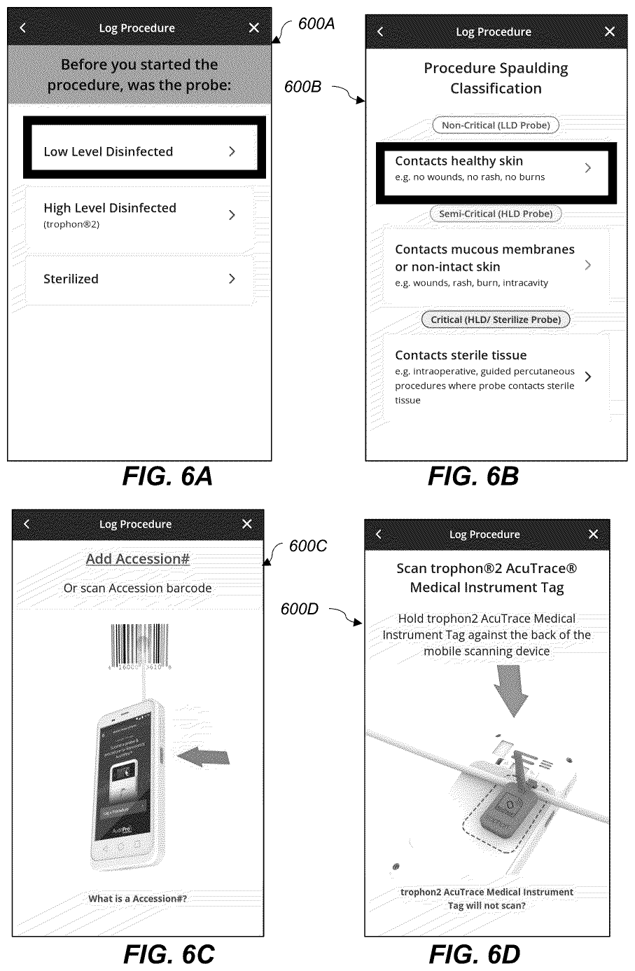

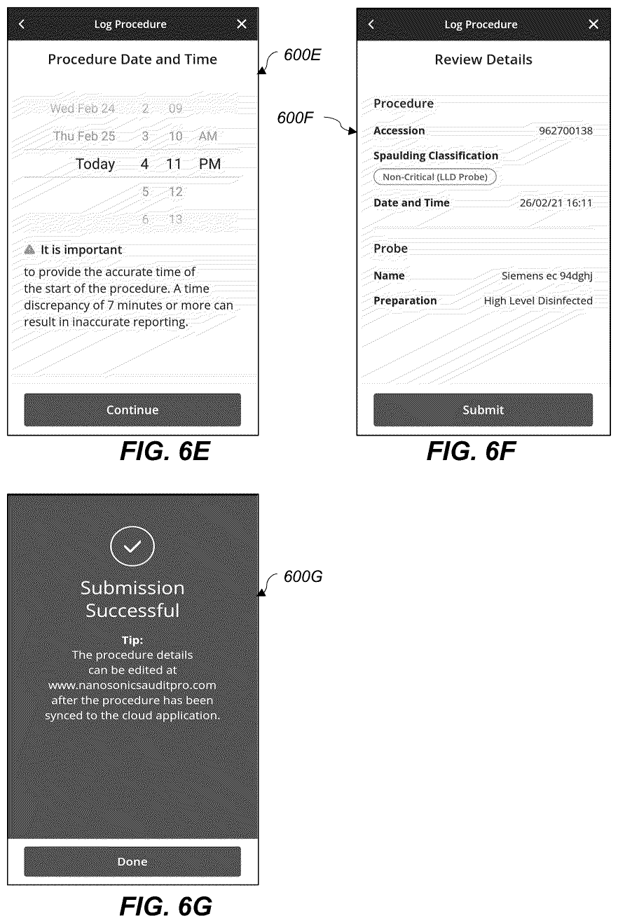

FIGS. 6A-6G are graphical user interfaces 600A-600G of a traceability and hygiene compliance management system for the reprocessing of reusable medical devices. In particular, FIGS. 6A-6G depict an example of logging a non-critical procedure. As depicted, logging a non-critical procedure requires that information about the procedure be provided by the user, followed by scanning the accession code of the procedure, scanning a code that identifies the reusable medical devices, and confirming the logged information. In particular, the information gathered shows that a low-level disinfection was performed, and the procedure Spaulding classification indicates contact of healthy skin.

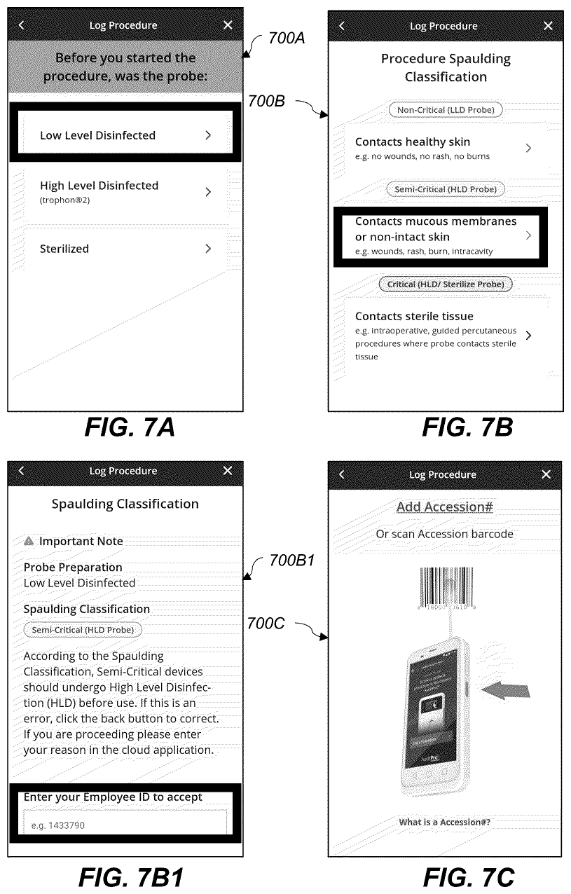

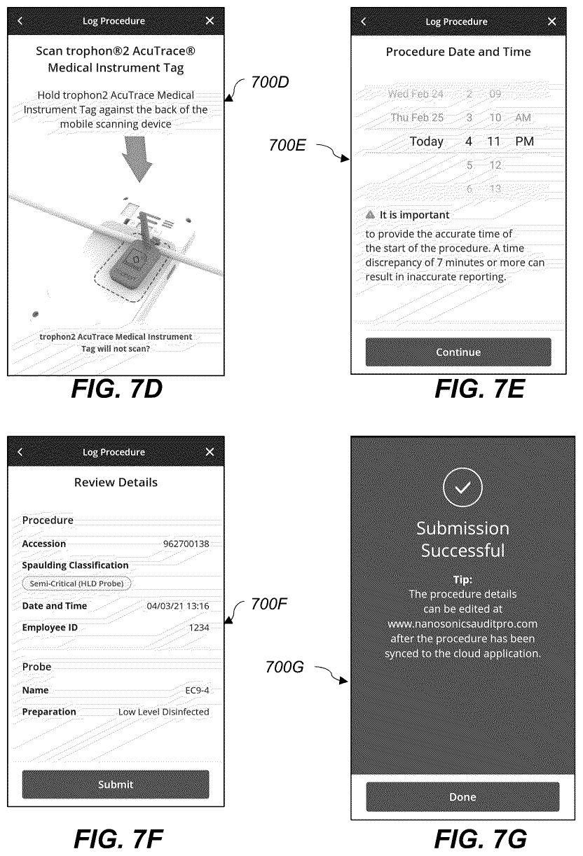

FIGS. 7A-7G are graphical user interfaces 700A-700G of a traceability and hygiene compliance management system for the reprocessing of reusable medical devices. In particular, FIGS. 7A-7G depict an example of logging a semi-critical procedure. In this example, additional information is required in order to validate the semi-critical procedure. In particular, the information gathered shows that a low-level disinfection was performed, and the procedure Spaulding classification indicates contact of mucous membranes or non-intact skin, leading to a prompt for employee ID to confirm the important note about probe preparation depicted in FIG. 7B1. In other embodiments, numerous different inquiries may be made by the user interface to collect other information related to a semi-critical procedure.

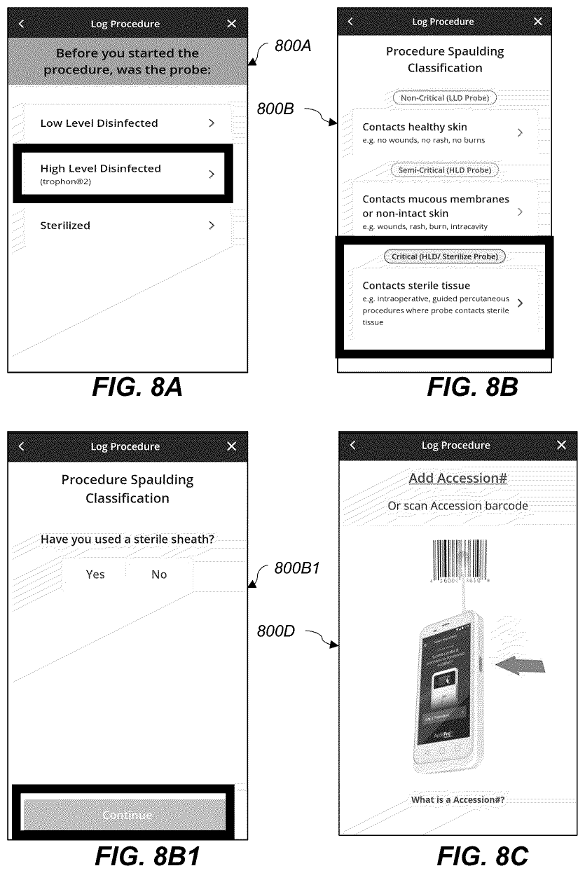

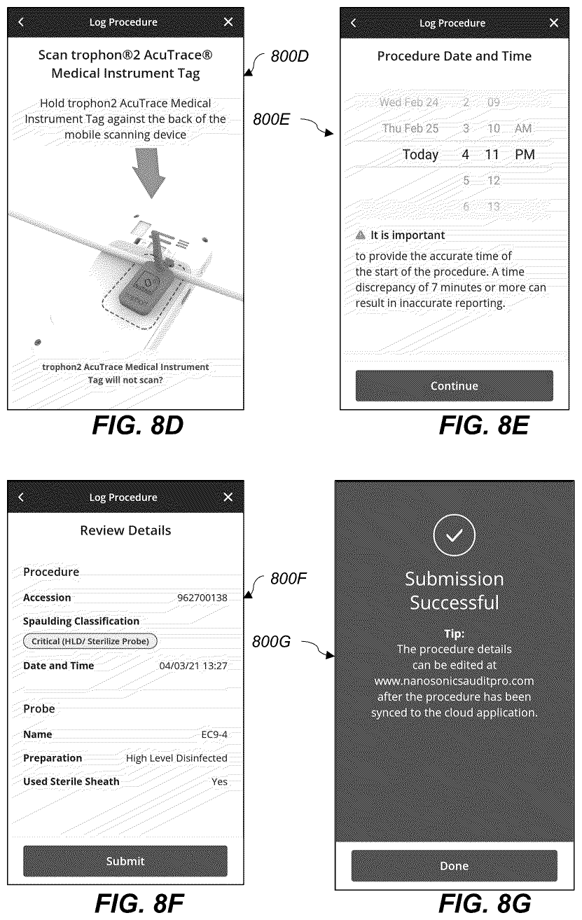

FIGS. 8A-8G are graphical user interfaces 800A-800G of a traceability and hygiene compliance management system for the reprocessing of reusable medical devices. In particular, FIGS. 8A-8G depict an example of logging a critical procedure that has been high-level disinfected with a probe cover (sterile sheath). In this example, additional information is required in order to validate the critical procedure. In particular, the information gathered shows that a high-level disinfection was performed, and the procedure Spaulding classification indicates contact of sterile tissue, leading to a prompt for further questions. First, the user interface at FIG. 8B1 inquires about the use of sterile sheath, and if the answer is yes, then proceeds to FIG. 8C. In other embodiments, numerous different inquiries may be made by the user interface to collect other information related to a critical procedure.

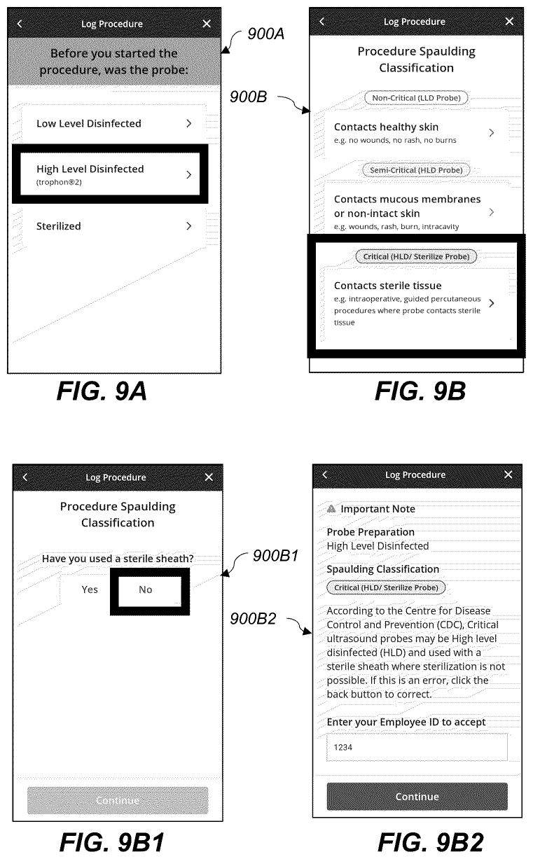

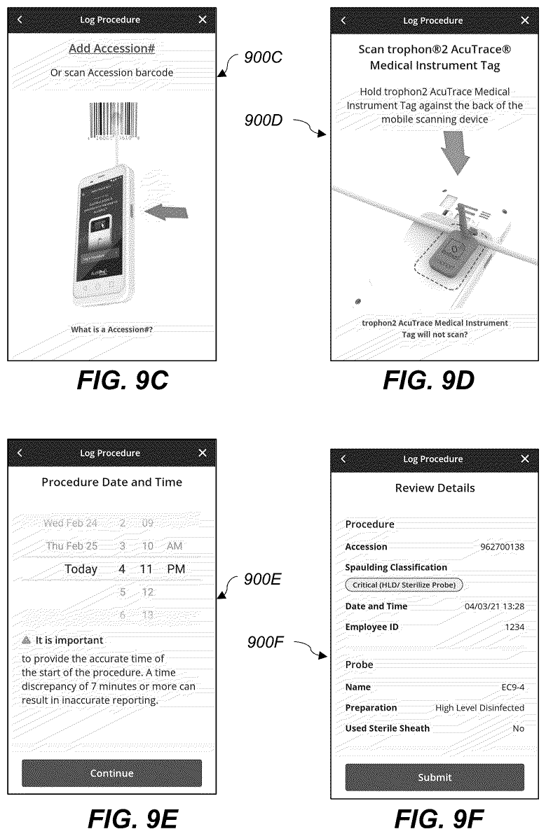

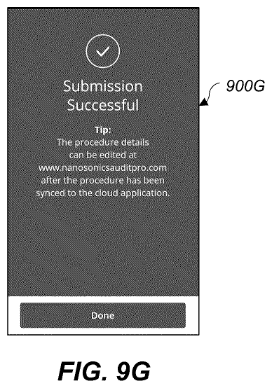

FIGS. 9A-9G are graphical user interfaces 900A-900G of a traceability and hygiene compliance management system for the reprocessing of reusable medical devices. In particular, FIGS. 9A-9G depict an example of logging a critical procedure that has been high-level disinfected without a probe cover (sterile sheath). In this example, additional information is required in order to validate the critical procedure. In particular, the information gathered shows that a high-level disinfection was performed, and the procedure Spaulding classification indicates contact of sterile tissue, leading to a prompt for further questions. First, the user interface at FIG. 9B1 inquires about the use of sterile sheath, and if the answer is no, then proceeds to FIG. 9B2 with a prompt for employee ID to confirm the important note about probe preparation depicted. In other embodiments, numerous different inquiries may be made by the user interface to collect other information related to a critical procedure.

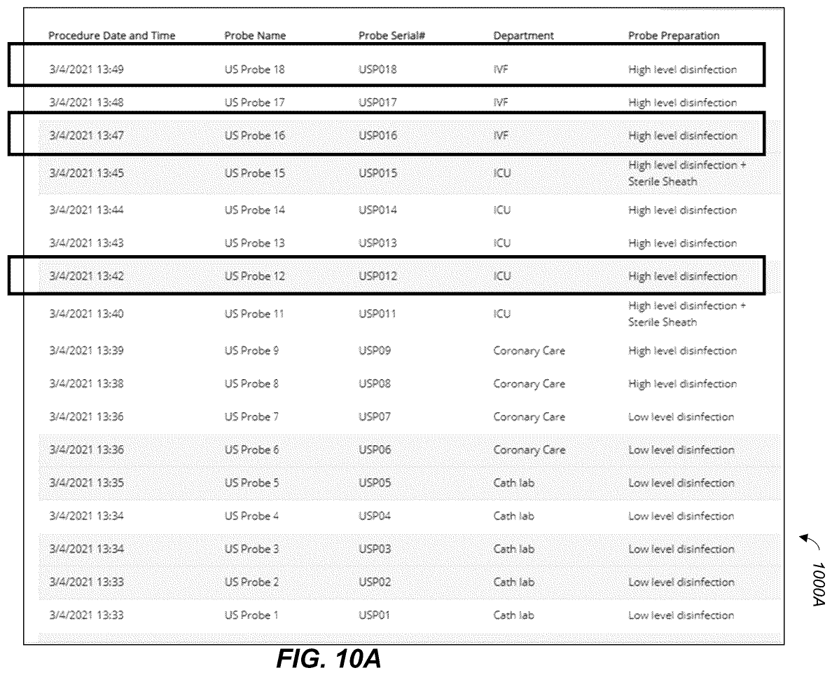

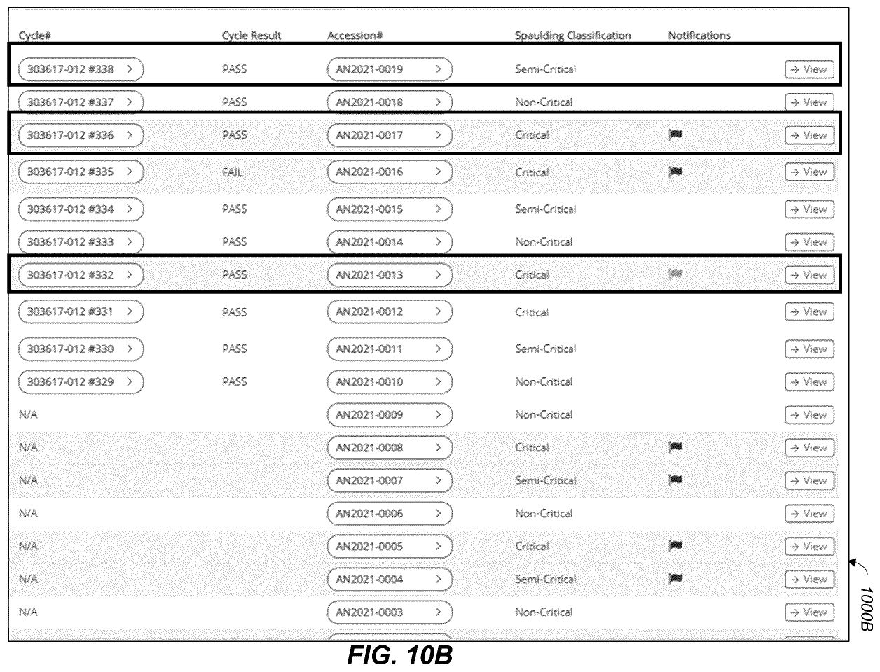

FIGS. 10A-10B are graphical user interfaces 1000A-1000B of a traceability and hygiene compliance management system for the reprocessing of reusable medical devices. In particular, FIGS. 10A-10G depict an example of different alerts that are generated in the system, with FIG. 10A being the left hand side of a graphical interface table and FIG. 10B being the right hand side of the graphical interface table. As shown in FIGS. 10A-10B, notifications have been generated based on the information gathered regarding procedures, disinfection, and patients. For instance, the fourth procedure listed is a critical procedure that has failed for probe serial number USP015. In one embodiment, the notification triggers information to cause reprocessing of the probe so that it will be available for further procedures.

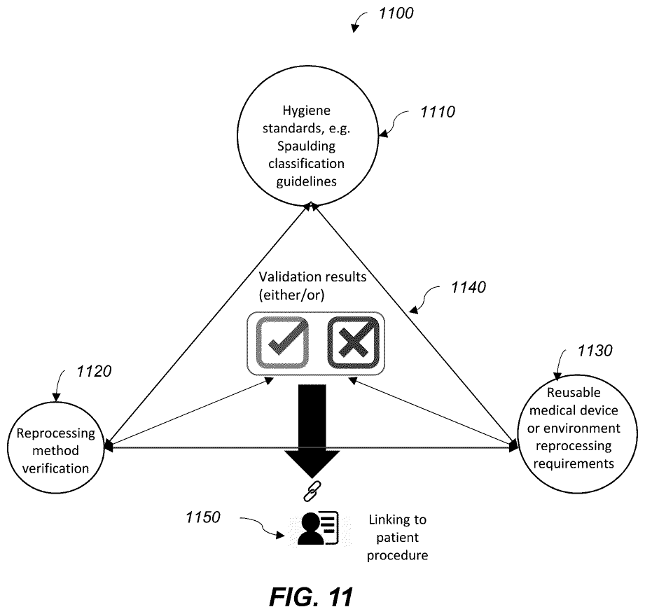

FIG. 11 is a flowchart summarizing a traceability method 1100 according to many embodiments of the present disclosure. In one or more embodiments, the system validates the safe reuse to hygiene standards of reusable medical devices and environments, and advantageously, the system links evidence based reprocessing to patient procedures to reduce patient cross contamination risk, provide evidence based reprocessing documentation and support risk mitigation through alert notifications of risk events, evidence based safe usage indications of devices and environments and data led insights into infection prevention practiced within medical environments and devices. Specifically, the method 1100 at block 1110 accounts for hygiene standards, e.g., Spaulding classification guidelines. The method 1100 at block 1120 performs reprocessing method validation. The method 1100 at block 1130 ensures reusable medical device or environment reprocessing requirements. The method 1100 at block 1140 validates this information to provide traceability as discussed above. The method 1100 at block 1150 links to patient procedures.

Thus, from a medical device perspective, as depicted in FIGS. 1-11, the present technique advantageously enables a clinician/cleaning technician at the point of care to identify the medical procedure classification or environment type, the reusable medical device unique ID to be used in the procedure or environment unique ID, the reprocessing action undertaken before the procedure (including any active agents or consumables used within the reprocessing process, critical parameters, contact times and other validation) and the patient procedure identifier to link reusable medical device reprocessing events to the specific patient and calculate a response to the compliance according to governing hygiene standards and guidelines. From a hygiene compliance standpoint, the integrated approach of the present technique allows for an advancement beyond the limitations of conventional techniques that do not bring together procedure and reprocessing information into specific actions taken to reduce cross-contaminations, etc.

For instance, if the log reductions resulting from the reprocessing event is inadequate to meet hygiene standards for this reusable medical device and procedure classification or the medical environment, the system of FIGS. 1-11 will advantageously alert the clinician/cleaning technician and remind them to meet the hygiene standards and guidelines governing jurisdiction, device and procedure classification before performing the procedure or the environment before making the environment available for the next visitors.

In addition, if the clinician/cleaning technician wish to accept this risk they will be required to log their employee ID and proceed to record this non-compliant event. Advantageously, as described in FIGS. 1-11, a non-compliant event will automatically trigger non-compliance notifications to supervisors in real-time to limit the exposure risk and assist in reprocessing compliance workflow management. The automated alerts can be used to take the medical device out of circulation to prevent cross-contamination.

Further, if the clinician/cleaning technician logs a compliant event, the system will advantageously collect, link and store validated medical device reprocessing cycles and events to provide document evidence in additional to the logic to ensure clinical and cleaning technician workflow compliance and accuracy to improve patient safety.

In one or more embodiments, as explained above with respect to FIGS. 1-11, logged information is collected through a range of mobile devices and in some instances from the reprocessing medical devices themselves, this information is sent to a cloud based application where it is manipulated to provide infection prevention risk management reports, reprocessing compliance reports, asset utilization and management reports, reprocessing reports, reprocessing personnel reports, digital logbook and other information to support best patient care. For instance, the above depicted centralized database will hold information uploaded from all reprocessing devices, Point of Care devices.

In additional, the technique of FIGS. 1-11 allows for environment reprocessing the evidence of reprocessing workflow compliance and essential pathogen log reduction required by selecting the environment type, the known contamination risk, the reprocessing action, the active agents utilized, cleaning technician ID, location ID. Cleaning technicians will scan in active agents, capture time stamped photograph to validate contact time and exposure or otherwise scan surfaces to show pathogen log reduction before and after reprocessing of the environment.

As a further explanation of the advantages of the techniques of FIGS. 1-11, the consideration starts with the patient procedure classification or environmental contamination risk, then triangulates the standards and guidelines requirements, the reusable device/environment, and the reprocessing log reduction requirements to provide validated evidence to confirm the reusable medical device or environment reprocessing workflow has been managed to governing laws, standards, guidelines and manufactures instructions to ensure patient protection.

Specific advantages realized in the technique of FIGS. 1-11 include the following:

The system will hold a database of hygiene standards and guidelines separated into specific details of procedure and environment classification requirements.

The system will hold a database of reusable medical devices and separate out each device's specific manufacture requirements for reprocessing, storage and usage and a system for linking the usage of these reusable medical devices to patient procedures and reprocessing events.

The system will hold a database of medical environments and the specific reprocessing requirements and have a method for capturing, validating, storing and linking reprocessing events to patient procedures and reprocessing events.

The system will hold a database of reprocessing medical devices and separate out their purpose, reusable device compatibility, log reduction capabilities and have a method for capturing, validating, storing and linking reprocessing cycles to patient procedures.

The system will hold a database of environmental reprocessing agents, activities and processes and have a method for capturing, validating, storing and linking reprocessing to specific environment and patient procedures.

The system will guide the clinician/cleaning technician at the point of care (POC) and in the environment through compliant reprocessing practices for the specific reusable medical device/environment, the procedure/environment classification and patient procedure/usage and capture validated evidence of compliance to pathogen log reduction to meet hygiene standards and guidelines relevant to the location of the facility and activity course correct malpractice through workflow management real time alerts and notifications to help reduce clinician and cleaning agent non-compliance and therefore patient risk of cross contamination;

This complex task will be achieved by identifying the standards and guideline jurisdiction, reusable medical device to be used OR the environment to be reprocessed, the procedure hygiene requirement classification OR the environmental contamination risk classification, the very specific reusable medical device manufacturer's instructions for reprocessing OR the specific environment reprocessing requirements, the reprocessing event date/time/operator/chemical agents/validation data. Using a complex algorithm the AP system will instruct clinicians and cleaning technicians through the reprocessing compliance workflow and alert them to non-compliant activities and alert supervisors and managers to risk events that have been confirmed either through clinician or cleaning technician acceptance or by the inability for the system to validate the required pathogen log reduction requirements to safely undertake a patient procedure or reuse an environment. Note that the requirement of reprocessing of environment example will be inverse to the reusable medical device in that the environment reprocessing need will depend on the contamination risk of the person/s prior, where are the reusable medical device reprocessing will depend on the exposure risk of the patient the device will be used on. For example, consider a vascular access scenario, where airborne bacteria could fall on the surface of the device and through contact pass into the blood stream to create a septic response.

Further advantages relate to patient acceptance. In one example of the present technique, the clinician can display to the patient the hygiene status of a reusable medical device prior to use. A patient viewing validation of hygiene data and digitally accepting the use of this device on their body would greatly enhance the relationship between the patient and the caregiver.

By way of summary, generally stated, described herein, in one embodiment, is a method for reusable medical device reprocessing compliance (e.g., compliance workflow management) and a system for implementing the method. For instance, the method includes the following steps: Reprocessing a reusable medical device before performing a second medical procedure and after performing a first medical procedure using a reprocessing procedure and uploading reprocessing data of the first reusable medical device. The hygiene status of the reusable medical device is validated before it is used on a patient to protect the patient from being cross contaminated. Reprocessing includes both the use of reprocessing devices, e.g., for disinfecting ultrasound probes, but also includes systems and equipment such as wipes, compliant chemical soaks/sprays that are applied using a chemical and a manual method for using such systems and equipment which will be validated by the method and system. Reprocessing could also include using a process that is consistent with manufacturer instructions. Performing the second medical procedure using the reusable medical device of the plurality of reusable medical devices and uploading first medical procedure data of the first medical procedure. Interactively validating the reusable medical device for a second medical procedure before performing the second medical procedure. The interactive validating comprises querying for procedure details of the second medical procedure and providing instructions based on the procedure details of the second medical procedure. Performing the second medical procedure using the reusable medical device and uploading second medical procedure data of the second medical procedure. Automatically generating an alert based on comparing the first medical procedure data of the first medical procedure, the procedure details and the second medical procedure data of the second medical procedure and the reprocessing data with a plurality of hygiene standards, the alert indicating noncompliance with at least one of the plurality of hygiene standards.

In one example, an alert is generated for the clinician before they use the reusable medical device advising them they are not meeting the standards. In another example, e.g., if the clinician accepts the risk of not meeting the standards, this logs the risk event and notifies the manager in real time for rapid risk assessment and course correction. For instance, alerts can be based on the identified hygiene status of the reusable medical device and the details of the second procedure. In addition, alerts can be based on noncompliance to manufacturer instructions (e.g., an ultrasound could be verified as available for reuse to hygiene standards having gone through a high temperature steamer, however this would damage the device and would go against the manufacturer's instructions for use and would thus raise an alert).

In one embodiment, the method further includes determining at least one other medical procedure performed using the reusable medical device and generating an alert identifying the at least one other medical procedure as having a risk of cross-contamination. In another embodiment, one or more mobile devices are used for the uploading of data to a central application, and the central application automatically generates the alert. In a further embodiment, the method further includes analysing past medical procedure and reprocessing data of the plurality of reusable medical devices to determine a reprocessing compliance baseline, the reprocessing compliance baseline comprising historical compliance levels of one or more standards, and generating alerts based on determining that future medical procedure and reprocessing data of the plurality of reusable medical devices deviate from the historical compliance levels. For instance, the use of wipes, chemical sprays, chemical soaks, etc., are validated based on compliance with relevant hygiene standards. Such methods may be validated by confirmation of how and when the method was performed, and relevant documentation thereof.

In one implementation, the method further includes analysing governing laws, standards, guidelines and instructions for use of reusable medical devices to determine a reprocessing compliance baseline. In another implementation, the reprocessing further includes a reprocessing device automatically uploading the reprocessing data. In a further embodiment, the reprocessing further includes the reusable medical device automatically uploading the reprocessing data.

In one example, the reusable medical device automatically uploads the first and second procedure data. In another example, the method further includes determining a risk to a second patient from a first patient due to, e.g., cross-contamination. In a further example, the reusable medical devices are mobile reusable medical devices.

In another aspect, the method and system demonstrate reusable medical device reprocessing compliance and/or environmental compliance (to relevant hygiene standards) to a patient. The method and system protect patients in healthcare environments who may be at risk of exposure of cross contamination despite their independent health state to manage the workflow compliance to better protect them from cross contamination. The method includes the following steps. Reprocessing a reusable medical device after performing a first medical procedure using a reprocessing procedure and uploading first reprocessing data of the reusable medical device. Interactively validating the reusable medical device for a second medical procedure before performing the second medical procedure, the interactive validating comprising querying for procedure details of the second medical procedure and providing instructions based on the procedure details of the second medical procedure. Receiving a validation message confirming that the reusable medical device complies with hygiene standards. Displaying the validation message to the patient. Performing the second medical procedure using the reusable medical device and uploading second medical procedure data of the second medical procedure. Automatically generating an alert based on comparing first medical procedure data of the first medical procedure, the procedure details and the second medical procedure data of the second medical procedure and the reprocessing data with a plurality of hygiene standards, the alert indicating noncompliance with at least one of the plurality of hygiene standards.

Embodiments are described herein with reference to flowchart illustrations and/or block diagrams of methods, apparatus (systems), and computer program products according to embodiments. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A method for reusable medical device reprocessing compliance management, the method comprising:
reprocessing a reusable medical device before performing a second medical procedure and after performing a first medical procedure using a reprocessing procedure and uploading first reprocessing data of the reusable medical device and the first medical procedure;
interactively validating the reusable medical device for the second medical procedure before performing the second medical procedure, the interactive validating comprising querying for procedure details of the second medical procedure and providing instructions based on the procedure details of the second medical procedure;
performing the second medical procedure using the reusable medical device and uploading second medical procedure data of the second medical procedure; and
automatically generating an alert based on comparing first medical procedure data of the first medical procedure, the procedure details and the second medical procedure data of the second medical procedure and the reprocessing and process validation with a plurality of hygiene standards, the alert indicating noncompliance with at least one of the plurality of hygiene standards.

2. The method of claim 1, further comprising determining at least one other medical procedure performed using the reusable medical device and generating an alert identifying at least one other medical procedure as having a risk of cross-contamination.

3. The method of claim 1, wherein one or more mobile devices are used for the uploading of data to a central application, and the central application or the mobile device automatically generates the alert.

4. The method of claim 1, further comprising analyzing past medical procedure and reprocessing data of the plurality of reusable medical devices to determine a reprocessing compliance baseline, the reprocessing compliance baseline comprising historical compliance levels of one or more standards, and generating alerts based on determining that future medical procedure and reprocessing data of the plurality of reusable medical devices deviate from the historical compliance levels.

5. The method of claim 1, further comprising analyzing governing laws, standards, guidelines and instructions for use of reusable medical devices to determine a reprocessing compliance baseline.

6. The method of claim 1, wherein the reprocessing further comprises a reprocessing device automatically uploading the reprocessing data.

7. The method of claim 1, wherein the reprocessing further comprises the automatically uploading the reprocessing data.

8. The method of claim 1, wherein performing the first and second procedures further comprises uploading the first and second procedure data.

9. The method of claim 1, wherein the first procedure data comprises first patient data and the second procedure data comprises second patient data, and the method further comprises determining a risk to a second patient of the second patient data from a first patient of the first patient data due to a cross-contamination.

10. The method of claim 1 wherein the reusable medical devices comprise mobile reusable medical devices.

11. A system for hygiene standards compliance of an environment or a device, the system comprising:
a central application;
one or more mobile devices, the one or more mobile devices configured to:
upload reprocessing data to the central application, the reprocessing data relating to a reprocessing procedure of the environment or the device;
upload first procedure data of a first procedure involving the environment or the device to the central application;
interactively validate the environment for a second procedure before performing the second procedure, the interactive validating comprising querying for procedure details of the second procedure and providing instructions based on the procedure details of the second procedure;
upload second procedure data of a second procedure involving the environment or the device to the central application,
wherein the central application automatically generates an alert based on comparing the first procedure data of the first procedure, the procedure details and the second procedure data of the second procedure and the reprocessing data with a plurality of hygiene standards, the alert indicating noncompliance with at least one of the plurality of hygiene standards.

12. The system of claim 11, wherein the environment comprises an emergency vehicle.

13. The system of claim 11, wherein the central application further determines at least one other procedure performed using the environment or the device and generates an alert identifying the at least one other procedure as having a risk of cross-contamination.

14. The system of claim 11, wherein the central application further analyzes governing laws, standards, guidelines and instructions for use of the environment or the device to determine a reprocessing compliance baseline.

15. The system of claim 11, the central application automatically receives the procedure data.

16. The system of claim 11, wherein the central application correlates historical procedure data to determine persons exposed to a hygiene risk by the noncompliance with the at least one of the plurality of hygiene standards.
